(19) United States
(12) Patent Application Publication
(10) Pub. No.: US 2010/0082034 A1
(43) Pub. Date: Apr. 1, 2010

(54) IMPLANT DESIGNS, APPARATUS AND METHODS FOR TOTAL KNEE RESURFACING

(76) Inventor: LEONARD REMIA, FT. Lauderdale, FL (US)

Correspondence Address: RICHARD S. ROSS, ESQ., 4801 S. UNIVERSITY DR., # 237, FT. LAUDERDALE, FL, 33328, US

(21) Appl. No.: 12/509,268

(22) Filed: Jul. 24, 2009

Related U.S. Application Data
(60) Provisional application No. 61/083,390, filed on Jul. 24, 2008.

(51) Int. Cl. A61B 17/58; A61F 2/38

(52) U.S. Cl. 606/88; 623/20.15; 623/20.21

(57) ABSTRACT

A ligament and bone conserving prosthesis for total knee resurfacing includes a distal femoral component which resurfaces the weight bearing portions of both femoral condyles and the trochlear groove. The prosthesis also includes implants to independently resurface the medial and lateral tibial plateaus in an inset manner. Also disclosed are apparatus and methods for performing the total knee resurfacing utilizing a minimally invasive, bone and ligament conserving manner.

### CROSS REFERENCE TO PROVISIONAL PATENT APPLICATION

Priority claimed to provisional application Ser. No. 61/083,390 filed Jul. 24, 2008.

### FIELD OF THE INVENTION

This invention relates to the field of minimally invasive joint resurfacing implants and to tools and techniques for their use. Among the preferred embodiments of the present invention are improvements in the design and deployment of total joint resurfacing implants and tools particularly applicable to orthopedic surgery and the treatment of osteoarthritic joints.

### BACKGROUND OF THE INVENTION

To provide prosthetic joint components for replacing damaged and deteriorating joints is well known. Typical joint replacements require resection of large amounts of bone from the end of one or more of the bones forming the joint to be replaced. Minimally invasive techniques and tools have been developed to aide in joint replacement procedures in an attempt to minimize soft tissue trauma and allow for a quicker functional recovery. However, the basic design and the amount of bone removed from the articular surfaces to allow placement of prosthetic total joint implants has not changed substantially. For example, in total knee replacement, a single implant covers or ‘replaces’ the entire articular portion of the femur and another implant, the entire articular portion of the tibia requiring transection of the Anterior Cruciate Ligament (ACL). Rather than decreasing the size of the implants (and in effect the amount of bone removed from the articular surfaces), surgical incisions have decreased due to the development of smaller instrumentation (minimally invasive techniques); however, current total knee technology requires removal of as much as 2 cm combined bone thickness from the articulating surfaces of the knee. Computer assisted surgery (CAS) has also been developed to aid in implant position, which can be difficult through a smaller incision, as visualization of bony structures and anatomical landmarks may be limited. Despite these advances, total knee replacement systems rely on complex metal jigs and intra-medullary alignment devices to ensure proper alignment of the implants to one another and to the articulating surfaces of the joint.

It may then be desirable to resurface only those articulating portions of the distal femur, specifically the medial and lateral condylar regions and the intervening trochlear groove, the corresponding medial and lateral articular portions of the tibia, and the patella. It may also be desirable to provide anatomically shaped implants for resurfacing of the articulating portions, to limit resection of ligaments and to preserve bone by insetting or inlaying the implants on the articular surfaces. The system may allow for resurfacing of only the weight bearing articular portion of the distal femur with a single implant and resurfacing of the medial and lateral articular portions of the tibia with two separate implants to preserve both the ACL and limit boney resection to as little as 3 mm from each articular surface. The articular portion of the patella may be also be resurfaced if significantly damaged.

It would then be advantageous to provide instruments for resecting the articulating surfaces of a bone to receive the resurfacing implants, that are minimally sized and that accurately guide a cutting tool to create curved inset surfaces for receipt of the implants. For example, U.S. patent Ser. No. 10/803,189 describes templates and milling devices for milling bone to a desired, standardized size and shape. However the invention provides a kit for partial knee replacement, but does not address total knee resurfacing.

It may also be desirable that the instruments for resecting the articular surfaces may be computer generated based on 3-D reconstruction data from either an MRI or CAT scan of the affected joint. Computer generated resecting guides allow for custom, anatomically matched fit to the articular surfaces of the joint, thus ensuring accurate bony resection and implant alignment. Implant alignment is ultimately responsible for longevity of total joint replacement; therefore, utilizing pre-operative computer assisted implant sizing, alignment and custom fit resection guides may prolong lifespan of the replacement.

### SUMMARY OF THE PRESENT INVENTION

Disclosed is a ligament and bone conserving prosthesis for total knee resurfacing. The prosthesis includes a distal femoral component, which resurfaces the weight bearing portions of both femoral condyles and the trochlear groove. The prosthesis also includes implants to independently resurface the medial and lateral tibial plateaus in an inset manner. Current tibial resection techniques for total knee replacement require resection of the entire medial and lateral tibial plateaus as a whole. The surgeon may elect to resurface the diseased patella as indicated.

Also disclosed are apparatus and methods for performing the total knee resurfacing utilizing a minimally invasive, bone and ligament conserving manner. Specifically, computer generated bone shaping guides provide a custom, accurate fit, allowing optimal alignment for each patient's knee. The bones are shaped following the normal curved bony anatomy and the prostheses are inset into the weight bearing portions of the distal femur and proximal tibia.

Also disclosed are standard, non-computer generated apparatus for resecting bone in the same manner as described in [007]. However the resecting guides may be a plurality of sizes to accommodate multiple curvatures, antero-posterior (AP) and media-lateral (ML) sizes of the articular surfaces of the knee.

Another objective is to describe the minimally invasive technique of total knee resurfacing as it relates to the disclosed prosthesis. Specifically, the surgical technique may utilize a single medial or medial and lateral incisions of the knee. These incisions are substantially smaller than standard joint replacement incisions and are designed to limit damage to the dermis and underlying tissues about the knee. The preceding descriptions are presented only as exemplary applications of the devices and methods according to the present invention. It should be understood that the detailed descriptions and specific examples, while indicating the preferred embodiment of the invention, are intended for purposes of illustration only, and are not intended to limit the scope of the invention.

### BRIEF DESCRIPTION OF DRAWINGS

FIG. 1 is a frontal (anterior) view of an embodiment of a metallic femoral total knee resurfacing implant according to the present invention.

FIG. 2 is a side (lateral) view of an embodiment of a metallic femoral total knee resurfacing implant according to the present invention.

FIG. 3 is a back (posterior) view of an embodiment of a metallic femoral total knee resurfacing implant according to the present invention.

FIG. 4 is a perspective view of an embodiment of a computer generated femoral routing guide according to the present invention.

FIG. 5 is a detailed cross-sectional view of an embodiment of a computer generated femoral routing guide and routing bit according to the present invention.

FIG. 6 is a perspective view of an embodiment of a computer generated tibial routing guide according to the present invention.

FIG. 7 is a detailed view of an embodiment of a computer generated tibial routing guide according to the present invention.

FIG. 8 is a detailed side (lateral) view of an embodiment of a computer generated tibial routing guide according to the present invention.

FIG. 9 is a detailed cross-sectional view of an embodiment of a computer generated tibial routing guide and routing bit according to the present invention.

FIG. 10 is a perspective view of an anatomy after bony resection according to the present invention.

FIG. 11 is a perspective view of an embodiment of a high molecular weight high molecular weight polyethylene tibial implant according to the present invention.

FIG. 12 is a perspective view of an embodiment of a high molecular weight polyethylene and metallic tibial implant according to the present invention.

FIG. 13 is a perspective view of an anatomy after placement of tibial implants according to the present invention.

FIG. 14 is a perspective view of an anatomy after placement of a femoral implant according to the present invention.

FIG. 15 is a perspective view of an embodiment of a metallic femoral sizing guide according to the present invention.

FIG. 16 is a perspective view of an embodiment of a metallic tibial sizing guide according to the present invention.

### DETAILED DESCRIPTION OF DISCLOSED EMBODIMENTS

The present invention may be understood more readily by reference to the following detailed description of the preferred embodiments of the invention and the examples included herein. However, before the preferred embodiments of the devices and methods according to the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

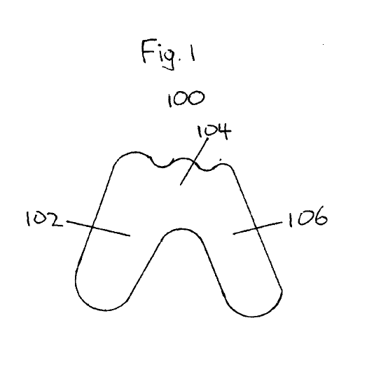

Referring now in more detail to the drawings, in which like numerals indicate like elements throughout the several views, FIG. 1 shows an embodiment of a metallic femoral total knee resurfacing implant 100 according to the present invention, comprising a lateral condylar flange 102, a medial condylar flange 106 and a trochlear flange 104. The implant 100 is seen here from its front (anterior) surface which closely mimics the distal articular surface of the femur.

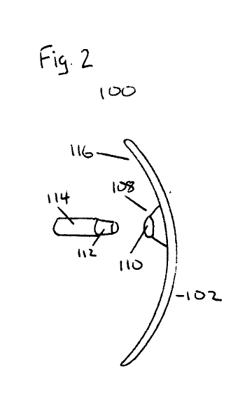

FIG. 2 is a side (lateral) view of an embodiment of a metallic femoral total knee resurfacing implant 100 according to the present invention, comprising a back (posterior) side 116 and a front side (denoted by the anterior portion of the lateral condylar flange 102) in the embodiment shown in FIG. 2, the posterior surface 116 includes a projection 108 containing a circular opening 110 continuous with a female cylindrical recess. The female cylindrical recess is tapered to accept the reverse tapered end 112 of a modular lug 114. The modular lug design allows for ease of implantation through smaller incisions during minimally invasive techniques.

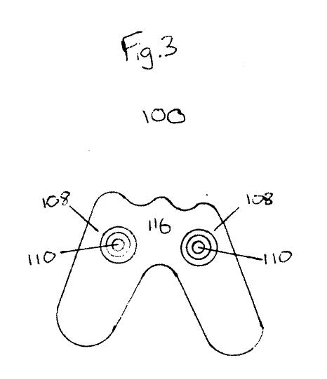

FIG. 3 is a back (posterior) view of an embodiment of a metallic femoral total knee resurfacing implant 100 according to the present invention, comprising a posterior surface 116 with two projections 108 containing said circular openings 110 continuous with female cylindrical tapered recesses. In one embodiment, the posterior surface 116 of the femoral implant 100 is roughened to accept bone cement. In another embodiment, the posterior surface 116 may be coated with a porous metallic substance to promote bony ingrowth.

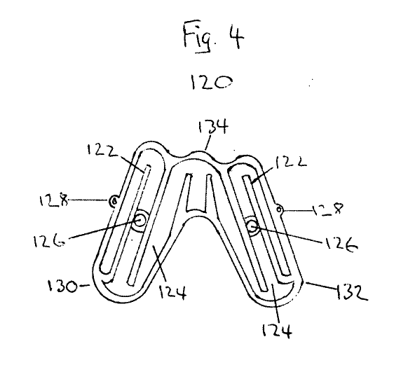

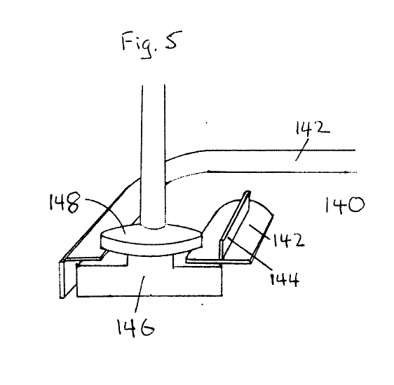

FIG. 4 is a perspective view of an embodiment of a computer generated femoral routing guide 120 according to the present invention comprising medial 132, lateral 130, and trochlear 134 portions. 3-D reconstructive data of the distal femur obtained from either an MRI or CAT scan are used to create the computer generated guide 120. The 3-D data is also used to pro-operatively template best fit size and alignment of the femoral implant 100 from FIG. 1, for the native anatomy. Utilizing a minimally invasive (3-5 inches) median parapatellar incision or a two incision technique comprising one 2 inch incision medial to and one 2 inch incision lateral to the patellar tendon, the articular surface of the femur is visualized. The femoral routing guide 120 is then secured to the distal femur with pins driven through the provided pin holes 128. The femoral routing guide 120 will anatomically match its back surface with the articular surface of the femur ensuring proper placement of the guide 120 and thus implant alignment. Multiple interconnected rails 122 form the tracts or paths 124 for the routing bit 146 shown in FIG. 5. Lug drilling guides 126 will accept a drill bit with an automatic depth stop to create lug holes in the distal femur, of matching diameter and depth as the modular lugs 114 shown in FIG. 3.

FIG. 5 is a detailed cross-sectional view of an embodiment of a computer generated femoral routing guide 140 and routing bit 146 according to the present invention. The rails of the guide 142 direct the path of the routing bit 146 with an integral depth stop 148 to accurately shape the bone to proper depth and shape. Raised styles 144 along the rails 142 help guide the routing bit 146 as well. The precise routing of the distal femur in 3 dimensions allows an accurate inset fit of the femoral implant 100 FIG. 1 to the level of the native articular surface. The femoral routing bit 146 may be attached in a plurality of ways to a handheld power tool which may rotate the bit 146 at a high RPM to resect bone accurately.

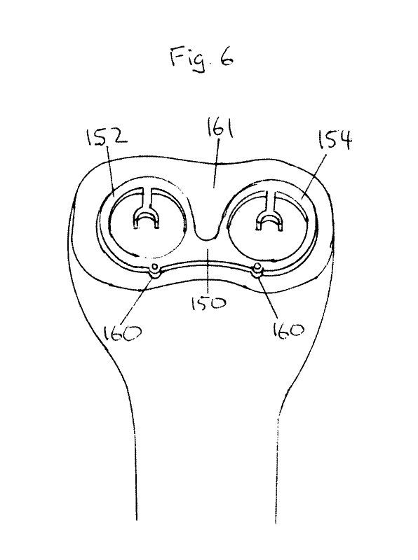

After preparing the distal femoral articular surface, the proximal tibia is addressed. Depending on the severity of osteoarthritic change, the medial tibial plateau alone, lateral tibial plateau alone, or medial and lateral tibial plateaus together may be prepared for resurfacing. FIG. 6 is a perspective view of an embodiment of a computer generated tibial routing guide 150 according to the present invention comprising medial 152 and lateral 154 circular tibial plateau resurfacing depth stop guides positioned over the proximal tibial articular surface 161. Pin holes 160 are located at the front (anterior) edge to help secure the guide 150 with metallic pins during routing.

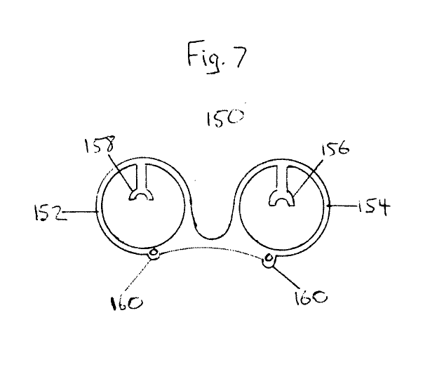

FIG. 7 is a detailed view of an embodiment of a computer generated tibial routing guide 150 according to the present invention comprising medial 152 and lateral 154 circular tibial plateau resurfacing guides and pin holes 160 on the front edge. Medial 158 and lateral 156 depth stop guides emanate from the posterior edge of the medial 152 and lateral 154 portions of the resurfacing guide 150.

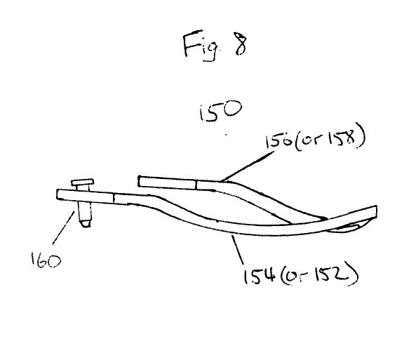

FIG. 8 is a detailed side (lateral) view of an embodiment of a computer generated tibial routing guide 150 according to the present invention. In this embodiment the medial 152 or lateral 154 portions of the guide 150 are shown from the side revealing a convex inferior surface anatomically matching the articular surface of the tibia. Seen projecting slightly superior to the guide itself are the depth stops 156 or 158 ending in a ‘U’ shaped configuration. The pin holes 160 along the front (anterior) edge of the guide 150 are also noted.

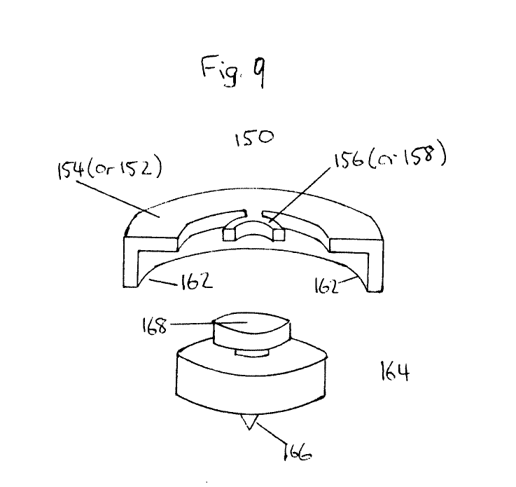

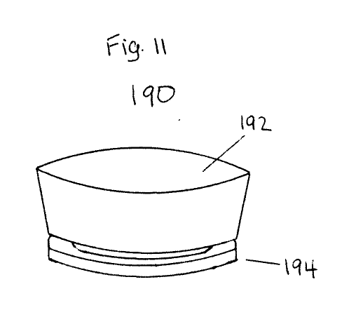

FIG. 9 is a detailed cross-sectional view of an embodiment of a computer generated tibial routing guide 150 and routing bit 164 according to the present invention. When preparing either the medial or lateral tibial plateaus, the routing bit 164 is prevented from penetrating too deeply into the bone by a collar 168 which comes to rest against the depth stops 156 and 158. The collar is calibrated to match the exact depth of the tibial implant 190 FIG. 11; the diameter of the routing bit 164 matches the diameter of the tibial implant 190 FIG. 11. The routing bits' 164 inferior side is covered with a pointed cutting surface 166. In this embodiment the routing bit 164 may be attached to in differing ways to a handheld power tool which may rotate the bit 164 at a high RPM to resect bone accurately.

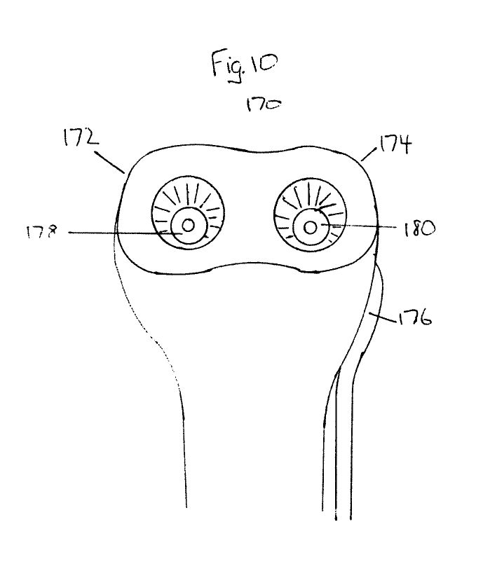

FIG. 10 is a perspective view of the proximal tibia 170 after bony resection according to the present invention comprising medial 172 and lateral 174 tibial plateaus. The medial 178 and lateral 180 routed female recesses are ready to accept the tibial implants 190 FIG. 11. The fibular head 176 is show for illustrative purposes.

FIG. 11 is a perspective view of an embodiment of a high molecular weight polyethylene tibial implant 190 according to the present invention. The superior 192 surface is slightly concave to match the curvature of the articular surface of the tibial plateau. In this embodiment the inferior part has a stepped recess to facilitate bone cement bonding. The “best fit” curvature of the superior 192 surface and the diameter of the implant 190 are determined preoperatively by 3-D reconstructive data and computer templating.

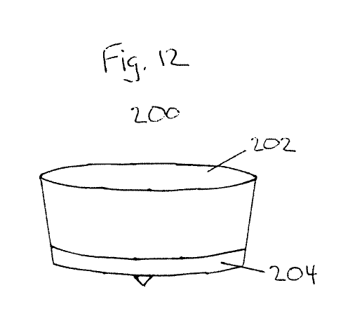

FIG. 12 is a perspective view of an embodiment of a high molecular weight polyethylene and metallic tibial implant 200 according to the present invention. In some cases such as osteoporotic bone it may be advantageous to utilize a metal backing 204. The metal backing 204 may add structural support to the polyethylene component 202 of the implant cement interface.

After preparation of the proximal tibial, the surgeon may elect to prepare for resurfacing of the posterior articular surface of the patella, depending on the severity of osteoarthritic disease. Then, the tibial components are generally implanted first, secured with bone cement. Next the femoral component is either cemented or press fit depending on bone quality and surgeon preference. Finally the patellar component is cemented into place.

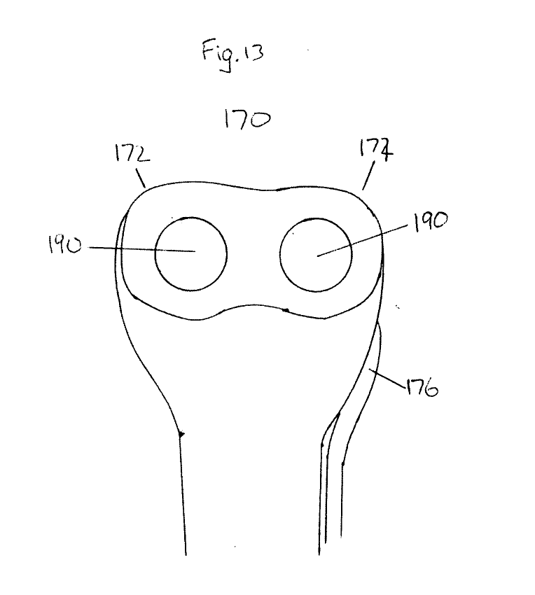

FIG. 13 is a perspective view of a proximal tibia 170 after placement of tibial implants 190 according to the present invention. The implants are inset within the medial 172 and lateral 174 tibial plateaus to the same level as the native remaining articular cartilage. The fibular head 176 is show for illustrative purposes.

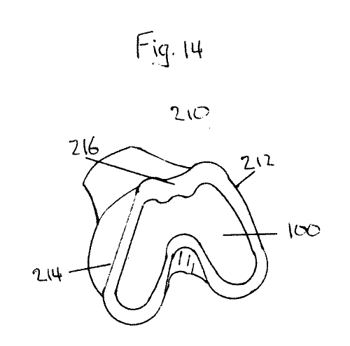

FIG. 14 is a perspective view of a distal femur 210 after placement of a femoral implant 100 according to the present invention. All three portions of the articular surface of the distal femur 210 are resurfaced including the medial femoral condyle 214, lateral femoral condyle 212 and trochlear groove 216.

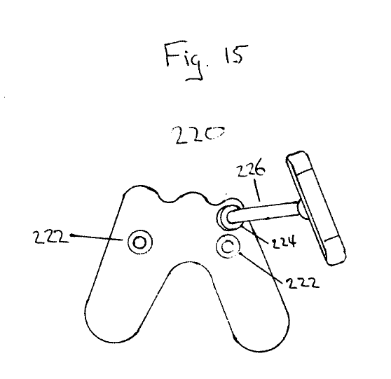

FIG. 15 is a perspective view of an embodiment of a metallic femoral sizing guide 220 according to the present invention comprising lug hole drill guides 220 and a detachable handle 226. In this embodiment, the sizing guide may be used instead of the computer generated routing guide 120 based on surgeon preference. A plurality of different sizes chosen to ‘best-fit’ the curvature, AP and ML size of the distal femur may be available as a kit. Once the femur is correctly sized and the lug holes drilled through the sizing guide, a metallic routing guide similar in design to the computer generated routing guide 120, is used to prepare the distal femur.

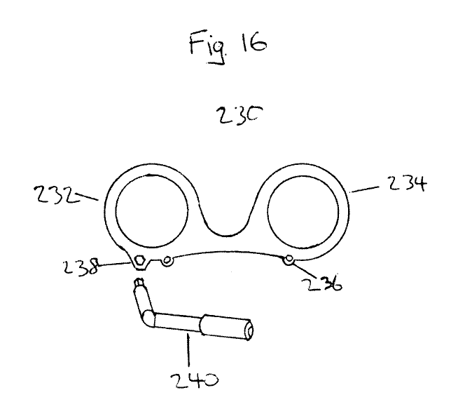

FIG. 16 is a perspective view of an embodiment of a metallic tibial sizing guide 230 according to the present invention comprising medial 232 and lateral 234 plateau sizing rings, a detachable handle 240 with corresponding attachment site 238, and pin holes 236. A plurality of different sizes chosen to ‘best-fit’ the curvature, AP and ML size of the proximal tibial may be available as a kit. Once the tibial plateaus are correctly sized the pin holes are drilled through the sizing guide and the guide then removed. A metallic routing guide similar in design to the computer generated routing guide 150, is used to prepare the proximal tibia.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the description and examples presented herein should not be construed to limit the scope of the present invention, the essential features of which are set forth in the appended claims.

We claim:

1. A prosthesis for total knee resurfacing consisting of a metallic femoral implant for fitting about both medial and lateral femoral condyles and the trochlear groove of the distal femur, modular lugs which attach the posterior aspect of the condylar components and are secured by a tapered fit. Said femoral implant resurfaces only the weight bearing portions of the distal femur utilizing an inset technique. Said femoral implant thus preserves bone and ligamentous structures of the knee.

2. Said femoral implant of claim 1 of which posteriorly attached lugs help secure the implant to the distal femur. Said femoral component and lugs can be secured to the distal femur either with bone cement or with a porous metal backing promoting bony ingrowth to the implant.

3. A prosthesis of claim 1 further comprising circular tibial implants composed of high molecular weight polyethylene (HMWPE) or a combination of HMWPE with a metallic backing or inferior surface. Said implants provide a bearing surface for the femoral implant. Said tibial implants can be used to resurface both weight bearing tibial plateau articular surfaces utilizing an inset technique

4. A computer generated femoral routing guide made to fit precisely and be removably attached to the distal femur. Said guide comprised of rails and styles which couple the motion of a bit to precisely shape the bone to accept the femoral implant.

5. A computer generated tibial routing guide made to fit precisely and be removably attached to the proximal ibia. Said guide comprised of rails and styles which couple the motion of a routing bit to precisely shape the bone to accept the tibial implants.

6. A method for minimally invasive total knee resurfacing; the method comprising the steps of:
Incising the dermis and underlying soft tissues either in a median parapatellar fashion or utilizing two smaller incisions, one on either side of the patellar tendon.
Fixing the removable computer generated femoral routing guide to the distal femur.
Interconnecting a routing bit within the channel formed by the rails of the routing guide and traversing the routing face with the routing tool to shape the bone.
Repeat similar steps for tibial routing guide.
Prepare posterior articular surface of patella as indicated
Cement tibial implants into weight bearing portions of tibial plateau.
Cement or press-fit femoral implant to distal femur. Lugs may be deployed first the attach femoral component to lugs; or lugs may be assembled onto femoral implant and deployed simultaneously.

7. The method of claim 6 wherein non-computer generated sizing and routing guides are utilized, per operator preference, to prepare the distal femur and proximal tibia for the resurfacing implants.
